(19) United States
(12) Patent Application Publication
(10) Pub. No.: US 2016/0355616 A1
(43) Pub. Date: Dec. 8, 2016

(54) Polyvinyl Pyrrolidone analog Polymers

(71) Applicant: Robert Bernard Login, Bothell, WA (US)

(72) Inventor: Robert Bernard Login, Bothell, WA (US)

(21) Appl. No.: 14/728,799

(22) Filed: Jun. 2, 2015

(51) Int. Cl. C08F 26/10; C07D 403/02

(52) U.S. Cl. CPC C08F 26/10; C07D 403/02

(57) ABSTRACT

N,N′-divinyldilactams, especially when the monomer is essentially two vinyl pyrrolidones linked together (Dimer Pyrrolidones). Said monomers are polymerized under anhydrous conditions in solvents or neat with HBr or by ADMET metathesis to useful polymers that have the ability to degrade under catalytic aqueous acidic conditions to small innocuous biodegrable compounds. Such polymers would be analogous to PVP and be easily excretable or biodegradable replacements.

This application claims the benefit of U.S. Provisional Patent Application Ser. No. 62/011,455 filed on Jun. 6, 2014.

### TECHNICAL FIELD AND BACKGROUND

This invention relates to novel easily degraded polymers especially those analogous to polyvinyl pyrrolidone (PVP). PVP is of significant value to society because of its remarkable properties of complexation, adhesion, film formation and very low toxicity; however, it cannot be degraded to fragments that are easily excreted and/or biodegraded when the polymer has done its job. Small compounds can be biodegrable or easily excreted from living organisms; therefore, such PVP analogs that have this property are of great utility. Polymers that mimic the performance of PVP but have said advantages are commercially unknown although several attempts to add hydrolysable groups to PVP's backbone have been reported. Apparently their hydrolyzed residual fragments are not sufficiently degradable or excretable; therefore, my embodiments would hydrolyze the PVP analog polymer all the way down to small compounds that could be easily biodegraded. The polymerization is not free radical (generating problem high MW polymers) and hence can be controlled to prepare polymers of low but effective molecular weight that could be excreted from human beings. Methods to synthesize a variety of said PVP analogs are the subject of this invention.

PVP was originally used during WW11 as a synthetic blood plasma expander by the Germans. It saved many lives because it was a universal blood substitute that would allow the victim many more hours of survival; time to reach help. After the war, the US stockpiled it in bomb shelters for the same reason. However, it was quickly realized that it was not excreted and caused long term problems. An excretable analog like those being claimed in this invention would be candidates for this important application not to mention the numerous other applications for NVP based polymers in pharmaceuticals, cosmetics, adhesives and so forth where a hydrolysable analog would be of value.

### DESCRIPTION OF THE INVENTION

### Acid Catalyzed Polymerization:

NVP (N-vinyl pyrrolidone) can be readily hydrolyzed in aqueous acid to 2-pyrrolidone, and acetaldehyde; however, under anhydrous conditions, acids like HBr will dimerize NVP. This reaction produces dimers in very high yield if not quantitatively. Other N-vinyl monomers prepared from various amides and imides are dimerized and also hydrolyzed in aqueous or mixed aqueous solvents when exposed to acidic conditions.

The following references describe this reaction in detail;
Huang et. al. Macromolecules, 2009, 42(21), pp 8198-8210: “Quantitative dimerization of NVP was also observed in the presence of ethyl 2-bromopropionate (EBP) and ethyl 2-bromoisobutyrate (EbiB) . . . .
   As proposed in Scheme 1, the bromoester-catalyzed dimerization of NVP could proceed via protonation of NVP with traces of HBr and dimer formation with another NVP unit, followed by release of HBr to start a new dimerization cycle.”

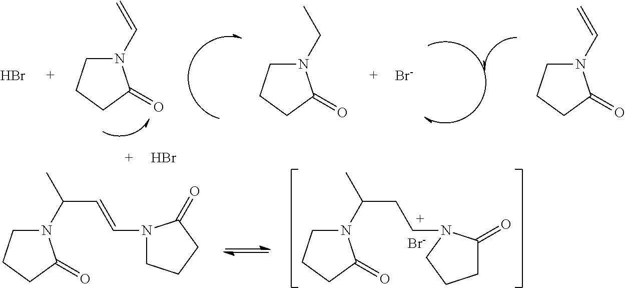

This dimer is also readily hydrolyzed in acidic aqueous solutions by the same mechanism as NVP. In fact residual NVP in polymers is eliminated by said hydrolysis. Therefore in one embodiment, N,N′ divinyl Dimer Pyrrolidone (or just Dimer Pyrrolidone) or dilactam monomers containing N,N′-divinyldilactam groups can be synthesized, followed by this anhydrous acid catalyzed reaction, resulting in a polymer with the pyrrolidone groups in the polymer backbone and would be analogous to PVP and have PVP like properties but would hydrolyze when exposed to aqueous acidic solutions. This is a unique unobvious reaction that form highly desirable polymers with the potential to solve current problems with PVP.

Madl and Spange, Macromolecules, 2000, 33, 5325-5335 “N-Methyl-N-vinylformamide and the cyclic N-vinylpyrrolidone (VP) dimerize with acidic catalysts, whereas N-methyl-N-vinylacetamide gives a mixture of low molecular products. The N-alkyl-substituted N-vinylamides are not able to undergo a oligomerization to higher oligomers (Mn>200) under the conditions investigated.”

As an alternative embodiment, said derivatives can also be polymerized by typical ADMET catalysts (Grubbs 1-3 or Schrock S—Mo) forming polymers by this metathesis reaction. (Chap. 7 in Olefin Metathesis Theory and Practice; Ed. K. Grela; Wiley, 2014 and chapter 1 in Advanced Polymer Science, Vol 176 by Baughman and Wagener.) This is illustrated below:

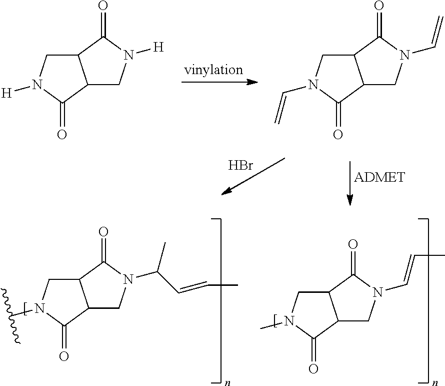 [SMILES: O=C1NCC2C(=O)NCC12 ; C=CN1CC2C(=O)N(C=C)CC2C1=O ; Br ; *N1CC2C(=O)N(C(C)/C=C/C)CC2C1=O ; C/C=C/N1CC2C(=O)N(C)CC2C1=O]

Polymers formed by either route in the presence of aqueous acidic solutions would hydrolyze to small compounds. In the case of the anhydrous acid catalyzed polymerization, this hydrolysis is identical to the known hydrolysis of said NVP dimer and is analogous to NVP itself. This reaction is illustrated below:

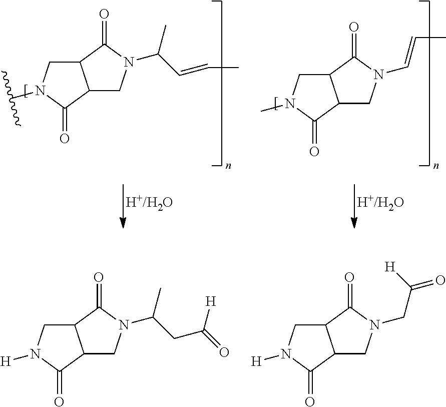

Furthermore, additional embodiments of said polymerization chemistry are applicable to other structures such as N,N′ divinyldiamides, or imides where after polymerization subsequent acid catalyzed hydrolysis would be of value. For example many plastics are resistant to being degraded to smaller compounds that would be considered innocuous or biodegradable; however, said acid catalyzed anhydrous polymerization of said divinyl monomers produces degradable plastics or resins when such polymers are exposed to acidic aqueous conditions. The resulting small residual compounds are much more vulnerable to biodegradation and this would be of immense value.

### PRIOR ART

F. Straub et. al., U.S. Pat. No. 4,254,239; Mar. 3, 1981; this patent employs low MW hydroxy terminated PVP chains extended with difunctional isocyanates. The idea is that such polymers would be vulnerable to hydrolysis and the fragments would be biodegradable. However, no known commercially available polymer resulted from this patent.

U.S. Pat. No. 5,912,312 (Zhong et. al. Jun. 15, 1999) illustrates how 2-methylene-1,3-dioxepane can be copolymerized with NVP to produce copolymers containing a multiplicity of backbone esters that are hydrolysable groups. The polymers are claimed to be biodegradable. However, sizeable amounts of this monomer are required thus diluting the PVP content. In addition, no commercial product to my knowledge has appeared based on this patent.

F. Becke and K. Wick, U.S. Pat. No. 3,037,001; May 29, 1962; describes the synthesis of various dilactams by the rearrangement of di-oximes in the Beckmann rearrangement. The dilactams are connected together by an R group. These dilactams could be easily vinylated and would be embodiments of my invention.

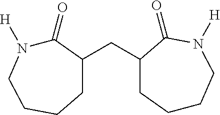 [SMILES: O=C1NCCCCC1CC1CCCCNC1=O]

N. M. Bortnick, U.S. Pat. No. 3,077,477; Feb. 12, 1963; describes the synthesis of bis-pyrrolidones from the reaction of ketonitriles and ketones in the presence of strong base catalysts. Said dipyrrolidones could be easily vinylated and would also be embodiments of my invention.

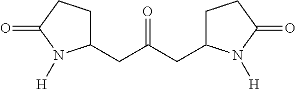 [SMILES: O=C(CC1CCC(=O)N1)CC1CCC(=O)N1]

W. L. Shilling, U.S. Pat. No. 3,235,562; Feb. 15, 1966; describes the reductive amination of dilevulinic acid with hydrogen and a catalyst to form 2,2-ethylenebis-(5-pyrrolidone).

This dilactone would be ideal for vinylation and is another embodiment of my invention.

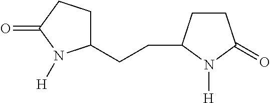 [SMILES: O=C1CCC(CCC2CCC(=O)N2)N1]

P. C. Wang, U.S. Pat. No. 4,847,388; Jul. 11, 1989; describes 1,6-diazaspiro[4,4]nonane which can be alkylated on the lactam nitrogen which indicates that this dilactam is ideal for vinylation and is another embodiment of my invention.

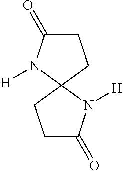 [SMILES: O=C1CCC2(CCC(=O)N2)N1]

A. Aumueller et. al., U.S. Pat. No. 5,008,312; Apr. 16, 1991; describes 2,8-dioxa-cis-bicyclo[3.3.0]octa-3,7-dione (see Tetrahedron 36 (1980), p 321 for synthesis) and its reaction with amines to produce dilactams suitable for my invention.

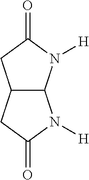

L. Hakanson and M. Heus, U.S. Pat. No. 6,458,980; Oct. 1, 2002 describes how to synthesize the bicyclic dilactam from 3,4-diamino-1,6-hexanedioic acid which was synthesized via the diamide of muconic acid (2,4-hexadiene-1,6-dioic acid) essentially as outlined by Kohl in Berichte 35, 173 (1903). In a modification of Kohl's procedure, the dimethylester of trans, trans-muconic acid was used. This is another example of an embodiment suitable for my invention.

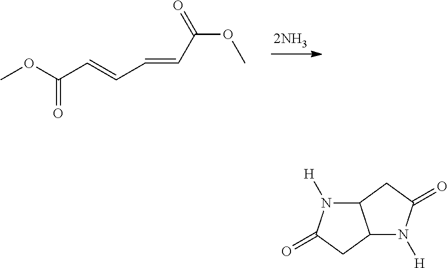

M. Connor et. al., USP 2006/0223993; Oct. 5, 2006; describes benzene-centered dilactams that could be vinylated and would produce polymerizable monomers of my invention.

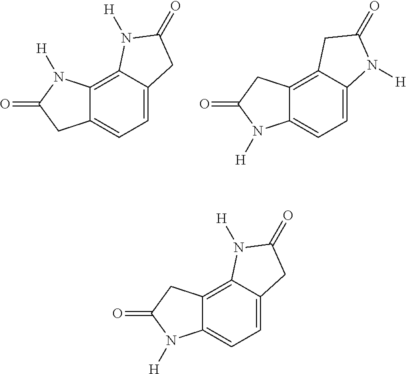 [SMILES: O=C1Cc2ccc3c(c2N1)NC(=O)C3 ; O=C1Cc2c(ccc3c2CC(=O)N3)N1 ; O=C1Cc2c(ccc3c2NC(=O)C3)N1]

### SUMMARY

Polymers of N,N′-divinyl monomers comprising those based on various N,N′-divinyl dilactams, diamides or diimides that can be polymerized with catalytic strong acids either neat or dissolved in suitable solvents under anhydrous conditions or by ADMET metathesis whereby said polymers are hydrolysable when dissolved in aqueous acidic solutions.

Examples of suitable Dimer Pyrrolidone monomers are illustrated below:

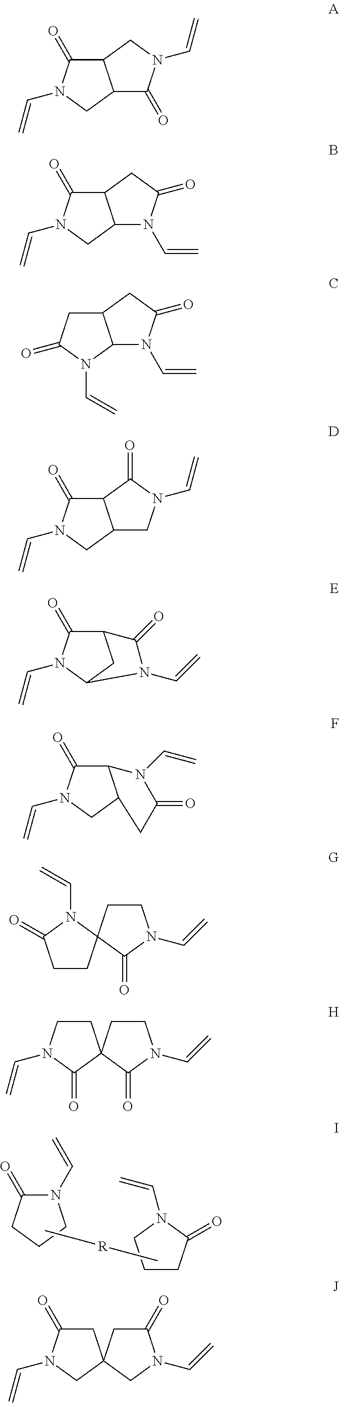

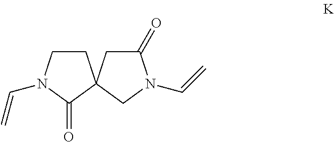

Wherein the R group connects two vinyl pyrrolidones at positions 3-5 which can be in any combination; and is an aliphatic chain of 1-10 carbon atoms, branched or linear, and can be optionally substituted with oxygen, nitrogen, aromatic or substituted aromatic rings.

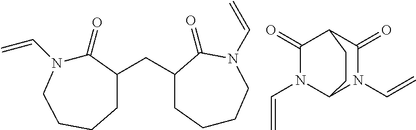

The above two examples would be embodiments of this invention but the optimum structures contain the desired Dimer Pyrrolidones.

However, the most useful examples are those that contain two vinyl pyrrolidones connected together (Dimer Pyrrolidones) as the object of this invention is to generate polymers analogous to PVP. The pyrrolidone group exhibits the greatest charge separation of the lactams with a negative charge on oxygen and a positive charge on nitrogen accounting for its water solubility and other desirable properties. For this reason many other vinyl lactams can be synthesized but have not been the billion dollar success of PVP.

The strong acid polymerization catalyst is chosen from mineral acids such as anhydrous HBr or HBr donors such as ethyl 2-bromopropionate(EBP) or ethyl 2-bromoisobutyrate (EBiB) and said polymerization would be conducted in an anhydrous solvent or neat.

The metathesis catalyst is chosen from those that are active in ADMET polymerizations such as the Grubbs 1-3 and Schrock S—Mo types.

The said polymers are hydrolyzed in acidic aqueous conditions.

Homopolymers or copolymers, prepared from N, N′ divinyl N-substituted amideimides, or N, N′ divinyldiimides and N, N′ divinyldilactams or mixtures of said monomers comprising the said anhydrous acid catalyzed polymerization or the ADMET metathesis catalyzed polymerization of said monomers affording polymers that are degraded by acid catalyzed hydrolysis. The optimum copolymers contain minor amounts of these other useful said divinyl types and the rest being Dimer Pyrrolidones.

Below are examples of these other monomers that are embodiments of this invention:

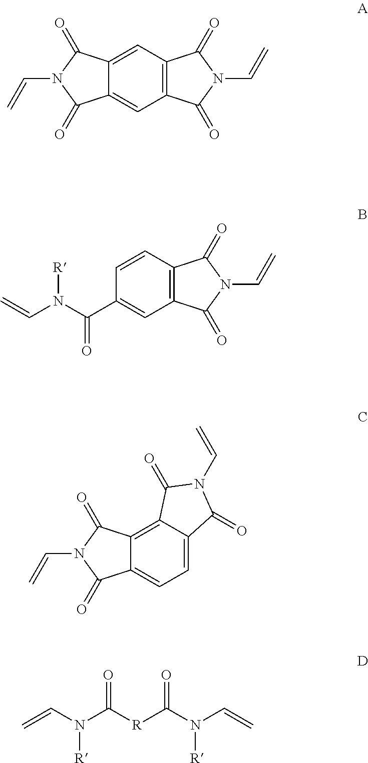

Wherein R′ is and alkyl group of 1-10 carbon atoms and R is a divalent alkyl group of 1-20 carbon atoms optionally substituted with aromatic moeities.

### DETAILED DESCRIPTION

Said high yield anhydrous acid catalyzed or ADMET metathesis reactions of said N,N′-divinyldilactam monomers would be ideal for polymerization but has never been proposed. Said monomers would contain the equivalent of two linked vinyl pyrrolidones (Dimer Pyrrolidones) or lactams. To synthesize said dimer lactone precursors, the most direct reaction is the unique one step high yield reaction of alkene and malonic acid in the presence of manganese(III)acetate:

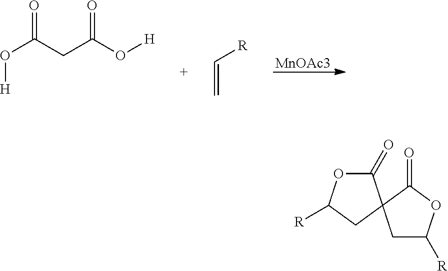

Fristad and Hershberger; JOC 1985, 50, 1026-1031
Jian-Qiang et. al. Chem J. of Chinese Universities, 2001, vol. 22, #5: 851-859.

### Also:

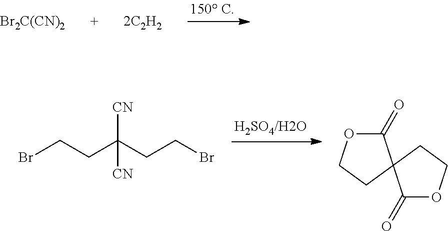 [SMILES: "Br2C(CN)2" ; N#CC(C#N)(CCBr)CCBr ; O=C1OCCC12CCOC2=O] [0036] J. R. Roland et. al.; JOC 1963, vol. 28, 2809-2811

The same type of spiro-dilactones can also be prepared from epoxides by the base catalyzed reaction with a maleate:

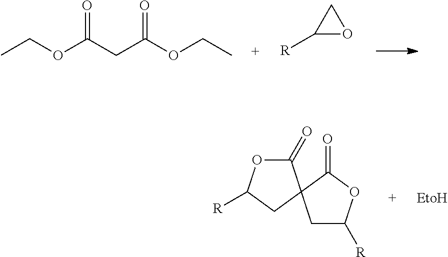

Rebrovic and Harris; U.S. Pat. No. 4,980,342
Leuchs and Gieseler. Ber., 1912, 45, 2114
Ishido et. al, J.C.S. Perkin I: 1977, 571-530.

Such spiro-dilactones are reacted with ammonia and the subsequent spiropyrrolidones vinylated by well known procedures. Such chemistry is carried out daily by a variety of large chemical companies in the manufacture of NVP and can be modified to work with said dimer pyrrolidones. A detailed review of the acetylene vinylation reaction and other methods of pyrrolidone vinylation can be found in Water Soluble Poly-N-Vinylamides by Y. E. Kirsh; Wiley, 1998, pp 3-16 which pages are referenced in their entirety.

The embodiments just described are known routes to several Dimer Pyrrolidones. Additional methods to synthesize said dimer lactams are proposed below but are not meant to be limiting. The route to each example is presented as a series of reactions that someone of ordinary skill in organic chemistry could follow to synthesize each example.

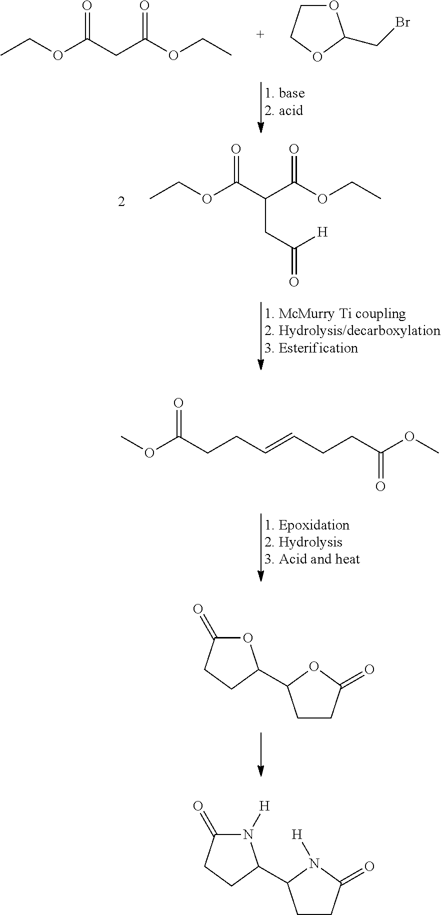

All of the raw materials for this synthesis are commercially available. Esters do not interfere with the McMurry coupling and conversion of unhindered olefins to diols can be accomplished by several routes.

The ethanolamine route to vinylation (see U.S. Pat. No. 7,141,679 B2; Kahn and references therein) would be more suitable in the laboratory for safety reasons but acetylene could be used industrially (As an aside, the dimer-lactone can also be condensed with various alkyl amines or diamines resulting in various derivatives with many useful properties. Long chain alkyl amines would result in gemini surfactants.).

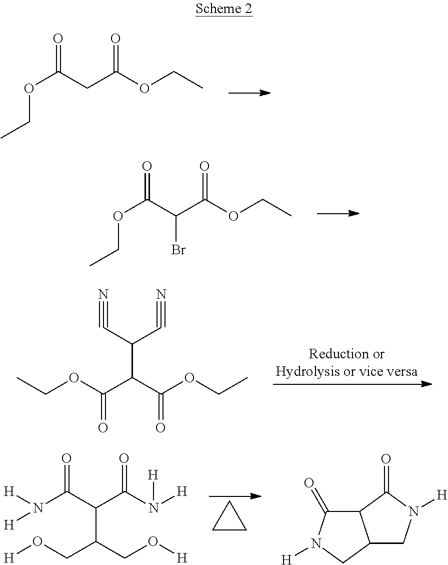

Diethyl 2-bromomaleate is commercially available as is malononitrile and esters are easier to reduce with hydrides than nitriles or (if hydrolyzed to amide) amides.

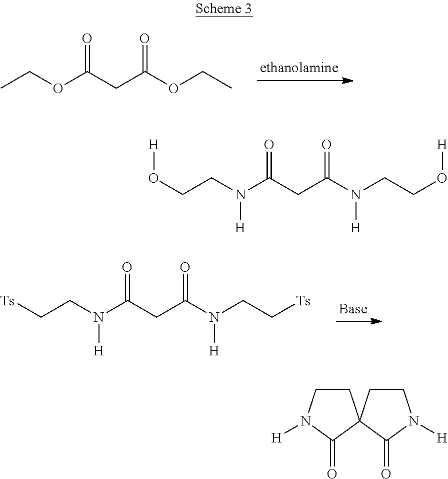 [SMILES: CCOC(=O)CC(=O)OCC ; O=C(CC(=O)NCCO)NCCO ; Cc1ccc(S(=O)(=O)CCNC(=O)CC(=O)NCCS(=O)(=O)c2ccc(C)cc2)cc1 ; O=C1NCCC12CCNC2=O]

Scheme 3 illustrates an alternative to the previously described reaction of maleic acid with olefins to produce the spiro-dilactone. Scheme 3 would be a more elegant approach to the said target molecule. Halides could also be substituted for the tosylate.

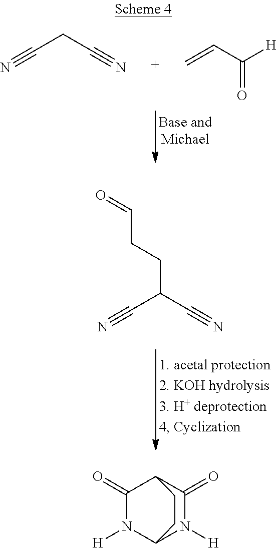

The Michael reaction of malononitrile with acrolien would be straight forward. Careful hydrolysis would be easily accomplished. The aldehyde can be protected as an acetal if that should prove to be necessary.

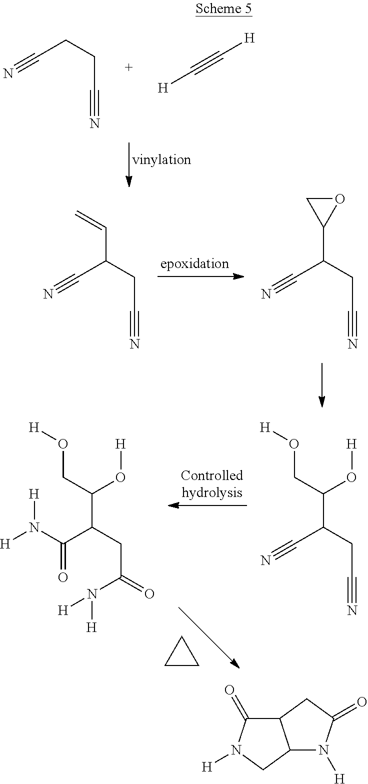

Succinonitrile could be vinylated with KOH as a catalyst. Various methods are known to epoxidize the olefin and convert it to a diol. Careful hydrolysis would produce the bis-amides and with heat convert them to lactams.

There are several methods of synthesizing the vinyl group for every said patent or other said examples and each method should be considered. There is an extensive patent literature concerning vinylation by the ethanolamine route (U.S. Pat. No. 6,906,200 B2, U.S. Pat. No. 7,141,679 B2 and many others) and this appears to be the preferred alternative to acetylene. It would be a safer method as the use of acetylene can be dangerous.

When in the presence of water, polymers prepared by said polymerization catalyzed by anhydrous HBr or ethyl 2-bromopropionate(EBP) or ethyl 2-bromoisobutyrate (EbiB) or by ADMET metathesis, will hydrolyze, when the pH is less than 7 in the presence of aqueous acids. At basic pH's, this hydrolysis would be suppressed; therefore, the breakdown of these polymers can be controlled at will and in addition said polymers would be water soluble, as the pyrrolidone group confers water solubility, another advantage. Said polymers and copolymers can be put to a variety of uses such as the delivery of poorly water soluble drugs, excipients such as tablet binders and disintegrants, cosmetic and personnel care additives such as hair care conditioners, thickeners, lubricants, mascaras and so forth. The advantage to the formulator is not only efficacy but the potential that the products after use could be easily removed forming innocuous byproducts. Also like pyrrolidone, said polymers would be expected to exhibit low skin and eye toxicity.

NVP and its polymers and copolymers have now been around essentially unchanged for more than fifty years. They are very valuable polymers but are not biodegradable or easily excreted from living beings. The polymers described in this invention would answer these problems and offer the possibilities of applications that once seemed so possible for PVP chief amongst them being as universal blood substitutes or expanders. This application never achieved success because of the poor degradation and elimination of PVP and the toxic effect of its remaining in the human body.

### EXPERIMENTAL

A. P. Kahn, U.S. Pat. No. 7,141,679 B2 (45); Nov. 28, 2006 describes how NVP is commonly manufactured by reacting 2-pyrrolidone with acetylene, but non-acetylenic routes to NVP are available. In one approach, NVP is made by dehydrating N-(2-hydroxyethyl)-2-pyrrolidone (HEP), which is easily obtained by reacting gamma-butyrolactone with 2-aminoethanol. The dehydrations are normally performed by passing a vaporized mixture of HEP (hydroxyethylpyrrolidone) and a diluent gas, usually nitrogen, through a heated reactor tube containing a catalyst. NVP is collected along with water, unreacted HEP, and by-products. There are many other patents describing the use of 2-aminoethanol (aka ethanolamine) that could be cited along with numerous patents describing other methods of vinylation.

### Example 1

### Preparation of a Divinyldilactam (as Per U.S. Pat. No. 6,906,200 to A. P. Kahn)

A tube (0.81″ ID With 0.185″ thermoWell) is packed with 10 cc (10.95 g) of Ca/Zn oxide (30 Wt. % Ca) that is ground to 14/30 mesh. The tube is heated in an electric furnace to 348° C. under a nitrogen flow of 18 SLH (standard liters per hour). N,N′ dihydroxyethyldipyrrolidone of patent U.S. Pat. No. 4,847,388 [N,N′-dihydroxyethyl-1,6-diazaspiro(4,4)nonane] is fed at 9.9 g/h to the top of the reactor and the product is recovered in an ice trap for analysis. After 10 hours, the corresponding N,N′-divinyl dipyrrolidone is obtained in good yield.

### Example 2

### Polymerization of the Bis-Vinyl Dilactam of Example 1 with HBr

Ten grams of the divinyldilactam of example 1 dissolved in 25 ml of toluene is added to a three neck RB flask equipped with mechanical stirrer, nitrogen sparge tube and a rubber septum. The charge is heated to 75 C and is then sparged for 15 minutes with anhydrous nitrogen. It is then mixed with 100 mg of anhydrous HBr which is slowly added through the septum by syringe. The mixture is mixed for two hours at 75-100 C where the viscosity is maximum and is then vacuum stripped of solvent at temperatures where the mixture is still liquid (up to 125 C) and is discharged into a polyethylene tray where it cools to a hard plastic. The product polymer forms stable solutions in dilute aqueous base but hydrolyses to low MW fragment in dilute aqueous mineral acids.

### Example 3

Repeat of example 2 but with ethyl 2-bromopropionate(EBP) initiator. Following exactly the same procedure, a very similar polymer is obtained.

### Example 4

### Polymerization of the Bis-Vinyl Dilactam of Example 1 with an ADMET Catalyst

In the same equipment as in example 2, ten grams of the same divinyldilactam is dissolved in ten ml of 1,2-dichloroethane. The mixture is heated to 50-60 C and sparged with anhydrous nitrogen for ten minutes, which is then converted to a nitrogen blanket. Grubbs type 1 catalyst (50 mg dissolved in 5 ml of dichloroethane) is added with stirring. After 3 hrs, there is no further increase in viscosity. The solvent is stripped off under vacuum and the molten polymer is discharged into a polyethylene tray where it cools to a plastic. This polymer forms stable solutions in dilute aqueous base but hydrolyses to low MW fragment in dilute aqueous mineral acids.

Although the invention has been described in terms of specific embodiments which are set forth in considerable detail, it should be understood that this description is by way of illustration only and that the invention is not necessarily limited thereto since alternative embodiments and operating techniques will become apparent to those skilled in the art in view of this disclosure. Accordingly, modifications are contemplated which can be made without departing from the spirit of this described invention.

1. A process for preparing polymers from N,N′-divinyldilactam monomers comprising:
exposing said monomers to a strong acid catalyst,
or an ADMET metathesis catalyst,
conducting said polymerization reaction neat or in solvents under anhydrous conditions,
heating to 40-125° C. or cooling to 0-25° C. said reaction mixture depending on the type of catalyst employed,
removing said solvent by vacuum stripping, and
optionally, neutralizing or removing said catalyst.

2. The process of claim 1 wherein the N,N′-divinyldilactam monomers are selected from the group consisting of dimer pyrrolidones compounds in which two pyrrolidones are connected together.

3. The process of claim 1 wherein the polymerization catalyst is selected from the group consisting of anhydrous HBr or HBr donors, ethyl 2-bromopropionate or ethyl 2-bromoisobutyrate.

4. (canceled)

5. The process of claim 1 wherein the polymers hydrolyze when exposed in solution at a pH of about 3 to 6.

6. (canceled)

7. Copolymers prepared by the the process, of claim 1, further comprising from 0.1 to 10 wt % of units derived from either N,N′-divinyldiamide, or N,N′-divinyldiimide or N,N′-divinylamideimide monomers.

8. The copolymers of claim 7 wherein said monomers other than said N,N′-divinyldilactams are selected from compounds represented by structures A-D. 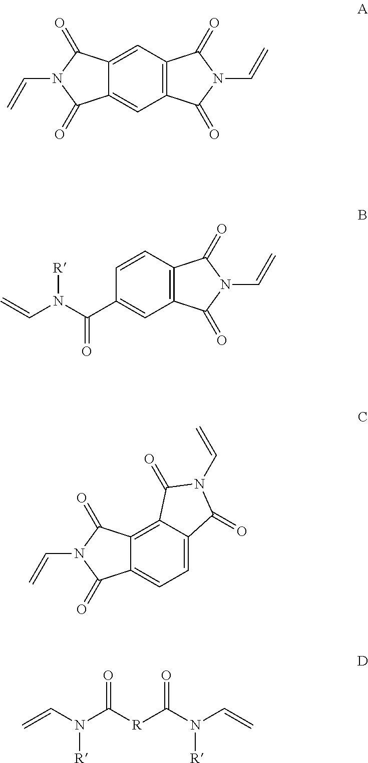
wherein R′ is an alkyl group of 1-10 carbon atoms and R is a divalent alkyl group of 1-20 carbon atoms optionally substituted with aromatic moieties.
